(19) United States
(12) Patent Application Publication
(10) Pub. No.: US 2010/0286208 A1
(43) Pub. Date: Nov. 11, 2010

(54) Novel polymorph of esomeprazole potassium and process for its preparation

(76) Inventors: Bobba Venkata Siva Kumar, Maharashtra (IN); Pravin BalachandrA Kulkarni, Kalyan (IN); Anil Ganpat Suryawanshi, Maharashtra (IN); Sachin Bapurao Sawant, Maharashtra (IN)

Correspondence Address: M. CARMEN & ASSOCIATES, PLLC, 1201 RXR Plaza, Uniondale, NY, 11556, US

(21) Appl. No.: 12/308,680

(22) PCT Filed: Jun. 21, 2007
(86) PCT No.: PCT/IB2007/001689

§ 371 Date: Jul. 12, 2010

Related U.S. Application Data
(60) Provisional application No. 60/858,098, filed on Nov. 10, 2006.

(30) Foreign Application Priority Data
Jun. 21, 2006 (IN) 971/MUM/2006

(51) Int. Cl. A61K 31/4439; C07D 401/12; A61P 29/00

(52) U.S. Cl. 514/338; 546/273.7

(57) ABSTRACT

Disclosed is a novel potassium salt of esomeprazole in polymorph form X, which can be used in pharmaceutical compositions. Processes for preparing polymorph form X of esomeprazole potassium are also provided.

### PRIORITY

This application claims the benefit under 35 U.S.C. §119 to U.S. Provisional Application No. 60/858,098, filed Nov. 10, 2006, and to Indian Provisional Application 971/MUM/2006, filed on Jun. 21, 2006, the contents of each of which are incorporated by reference herein.

### BACKGROUND OF THE INVENTION

1. Technical Field

The present invention generally relates to a novel crystalline form of 5-methoxy-2-[(S)-(4-methoxy-3,5-dimethyl-2-pyridinylmethyl) sulfinyl]-1H-benzimidazole potassium salt (also known as esomeprazole potassium), process for its preparation and pharmaceutical compositions containing same.

2. Description of the Related Art

The compound 5-methoxy-2-[[(4-methoxy-3,5-dimethyl-2-pyridyl)methyl]sulfinyl]-1H-benzimidazole, having the generic name omeprazole, and therapeutically acceptable salts thereof are well known as gastric acid secretion inhibitors, and are useful as anti-ulcer agents. The compound, being a sulfoxide, has an asymmetric center in the sulfur atom and may exist as a racemic mixture of its two single enantiomers, the R-omeprazole and the S-omeprazole. The optical isomers of omeprazole, particularly the (S) isomer, are believed to possess certain advantages over the racemic form. The absolute configurations of the enantiomers of omeprazole have been determined by an X-ray study of an N alkylated derivative of the (+)-enantiomer in neutral form. The (+)-enantiomer of the neutral form and the (−)-enantiomer of the neutral form were found to have the R and S configuration, respectively. The conditions for the optical rotation measurement for each of these enantiomers are described in, for example, WO 94/27988.

The enantiomer (S)-omeprazole is commonly referred to as esomeprazole (also known as (5-methoxy-2-[(S)-[(4-methoxy-3,5-dimethyl-2-pyridyl)methyl]-sulfinyl]-1H-benzimidazole-1-yl) and is represented by the structure of Formula I:

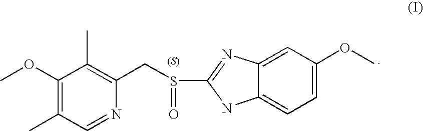

Esomeprazole is marketed in the United States as the magnesium trihydrate salt under the name Nexium® and is indicated for short-term treatment in the healing and symptomatic resolution of diagnostically confirmed erosive esophagitis. See, e.g., The Merck Index, Thirteenth Edition, 2001, pp. 1224-25, monograph 6913; and Physician's Desk Reference, “Nexium,” 58th Edition, pp. 624-28 (2004).

Certain salts of the single enantiomers of omeprazole and their preparation are disclosed in WO 94/27988. These compounds have improved pharmacokinetic and metabolic properties which will give an improved therapeutic profile such as a lower degree of inter individual variation.

WO 96/02535 discloses a process for the preparation of the single enantiomers of omeprazole and structurally related compounds as well as salts thereof WO 96/01623 discloses pharmaceutical dosage forms comprising for instance magnesium salts of (R)- and (S)-omeprazole.

WO 98/54171 discloses a process for the preparation of the trihydrate of magnesium salt of esomeprazole, wherein the potassium salt of esomeprazole is used as an intermediate.

U.S. Pat. Nos. 6,511,996 and 6,677,455 disclose two different crystalline forms of a potassium salt of esomeprazole.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Since the solubility of each polymorph may vary, identifying the existence of pharmaceutical polymorphs is essential for providing pharmaceuticals with predicable solubility profiles. It is desirable to investigate all solid state forms of a drug, including all polymorphic forms, and to determine the stability, dissolution and flow properties of each polymorphic form. Polymorphic forms of a compound can be distinguished in a laboratory by X-ray diffraction spectroscopy and by other methods such as, infrared spectrometry. Additionally, polymorphic forms of the same drug substance or active pharmaceutical ingredient, can be administered by itself or formulated as a drug product (also known as the final or finished dosage form), and are well known in the pharmaceutical art to affect, for example, the solubility, stability, flowability, tractability and compressibility of drug substances and the safety and efficacy of drug products.

The discovery of new polymorphic forms of a pharmaceutically useful compound provides a new opportunity to improve the performance characteristics of a pharmaceutical product. It also adds to the material that a formulation scientist has available for designing, for example, a pharmaceutical dosage form of a drug with a targeted release profile or other desired characteristic. A new polymorphic form of the potassium salt of esomeprazole has now been discovered and has been designated as Form X.

### SUMMARY OF THE INVENTION

In accordance with one embodiment of the present invention, a potassium salt of esomeprazole in polymorph form X is provided.

In accordance with a second embodiment of the present invention, a potassium salt of esomeprazole in polymorph form X and having an X-ray diffraction (XRD) pattern substantially in accordance with FIG. 1 is provided.

In accordance with a third embodiment of the present invention, a potassium salt of esomeprazole in polymorph form X and exhibiting a characteristic peak (expressed in degrees 2θ±0.2° θ) at about 5.3 is provided.

In accordance with a fourth embodiment of the present invention, a potassium salt of esomeprazole in polymorph form X having a differential scanning calorimetric (DSC) thermogram substantially in accordance with FIG. 2 is provided.

In accordance with a fifth embodiment of the present invention, a potassium salt of esomeprazole in polymorph form X and having a predominant endotherm peak at about 208.3° C. as measured by a Differential Scanning Calorimeter at a scan rate of 10° C. per minute with an Indium standard is provided.

In accordance with a sixth embodiment of the present invention, a potassium salt of esomeprazole in polymorph form X and having a thermograviometric (TGA) curve substantially in accordance with FIG. 3 is provided.

In accordance with a seventh embodiment of the present invention, a potassium salt of esomeprazole in polymorph form X and having temperature rise of 5° C./min in the range of about 30° C. to about 300° C. is provided.

In accordance with an eighth embodiment of the present invention, a pharmaceutical composition is provided comprising a therapeutically effective amount of a potassium salt of esomeprazole in polymorph form X.

In accordance with a ninth embodiment of the present invention, a process for preparing esomeprazole potassium in polymorph form X is provided, the process comprising:

(a) dissolving neutral esomeprazole in a first solvent;

(b) adding a second solvent to the solution;

(c) adding a source of potassium to the product of step (b); and

(d) recovering the esomeprazole potassium salt in polymorph form X.

In accordance with a tenth embodiment of the present invention, a process for preparing esomeprazole potassium in polymorph form X is provided, the process comprising:

(a) providing a solution of esomeprazole in a first solvent;

(b) adding a second solvent to the solution obtained in step (a);

(c) treating the solution of step (b) with a source of potassium;

(d) concentrating the reaction mass of step (c) under vacuum;

(e) adding a suitable anti-solvent; and

(f) recovering esomeprazole potassium in polymorph form X.

In accordance with an eleventh embodiment of the present invention, a process for preparing esomeprazole potassium in polymorph form X is provided, the process comprising:

(a) providing a solution of esomeprazole in a water immiscible organic solvent;

(b) adding a source of potassium to the solution to obtain a biphasic reaction mixture;

(c) separating the aqueous layer from the biphasic reaction mixture and removing water thereof to obtain a residue;

(d) optionally subjecting the residue to stripping to remove water; and

(e) recovering esomeprazole potassium in polymorph form X.

In accordance with a twelfth embodiment of the present invention, a method for reducing gastric acid secretion in a subject is provided which comprises administering to the subject an amount of the esomeprazole potassium in polymorph form X in solid form and effective to reduce gastric acid secretion by the subject.

### DEFINITIONS

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means an amount of the polymorph form X of esomeprazole potassium that is effective when administered alone or in combination to treat a state, disorder or condition, of a mammal and is sufficient to effect such treatment. The “therapeutically effective amount” will vary depending on the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “polymorph” as used herein refers to crystalline forms having the same chemical composition but different spatial arrangements of the molecules, atoms, and/or ions forming the crystal.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION OF THE DRAWINGS

FIG. 1 is a characteristic powder X-ray diffraction (XRD) pattern of polymorph form X of esomeprazole potassium.

FIG. 2 is a characteristic differential scanning calorimetric (DSC) thermogram of polymorph form X of esomeprazole potassium.

FIG. 3 is a characteristic thermo gravimetric analysis (TGA) of polymorph form X of esomeprazole potassium.

### DETAILED DESCRIPTION OF THE INVENTION

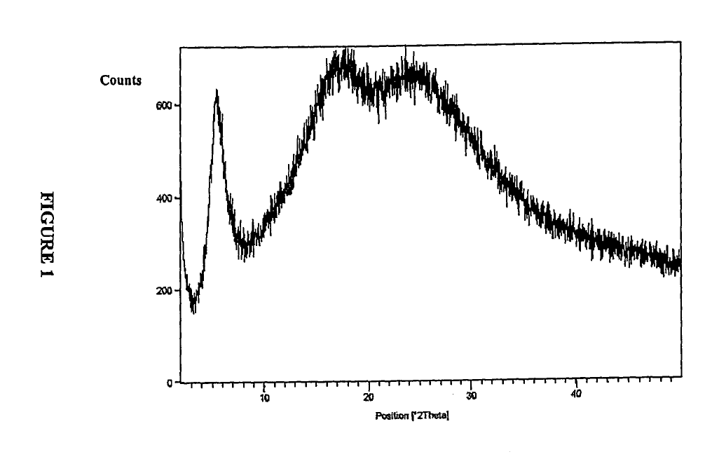

One embodiment of the present invention is directed to a novel polymorph form of a potassium salt of esomeprazole, designated polymorph form X. It has surprisingly been found that the potassium salt of esomeprazole occurs in a number of structurally different forms. The novel polymorph form X of esomeprazole potassium may be characterized by, for example, X-ray powder diffraction pattern and/or melting point. The powder XRD spectrum for polymorph form X is presented in FIG. 1, and is generally set forth in tabular form in Table I below. The X-Ray powder diffraction can be measured by an X-ray powder Diffractometer equipped with a Cu-anode (λ=1.54 Angstrom), X-ray source operated at 45 kV, 40 mA and a Ni filter is used to strip K-beta radiation. Two-theta calibration is performed using an NIST SRM 640c Si standard. The sample was analyzed using the following instrument parameters: measuring range=2-50° 2θ; step width=0.017°; and measuring time per step=5 sec.

TABLE I

| d-spacing | 2 - Theta | Relative Intensity (%) |
|---|---|---|
| 16.6674 | 5.3 ± −0.2 | 100 |

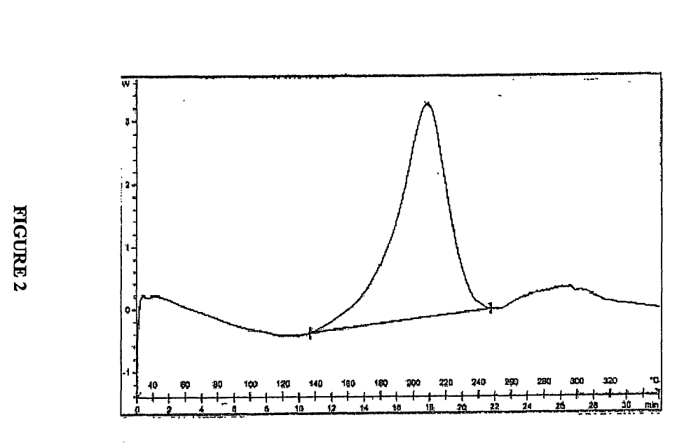

As shown in FIG. 2, polymorph form X of esomeprazole potassium exhibits a predominant endotherm peak at about 208.3° C. as measured by a Differential Scanning Calorimeter (DSC 822, Mettler Toledo) at a scan rate of 10° C. per minute with an Indium standard. In this regard, it should be understood that the endotherm measured by a particular differential scanning calorimeter is dependent upon a number of factors, including the rate of heating (i.e., scan rate), the calibration standard utilized, instrument calibration, relative humidity, and upon the chemical purity of the sample being tested. Thus, an endotherm as measured by DSC on the instrument identified above may vary by as much as ±1° C. or even ±2° C. Accordingly, the term “about 208.3” is intended to encompass such instrument variations.

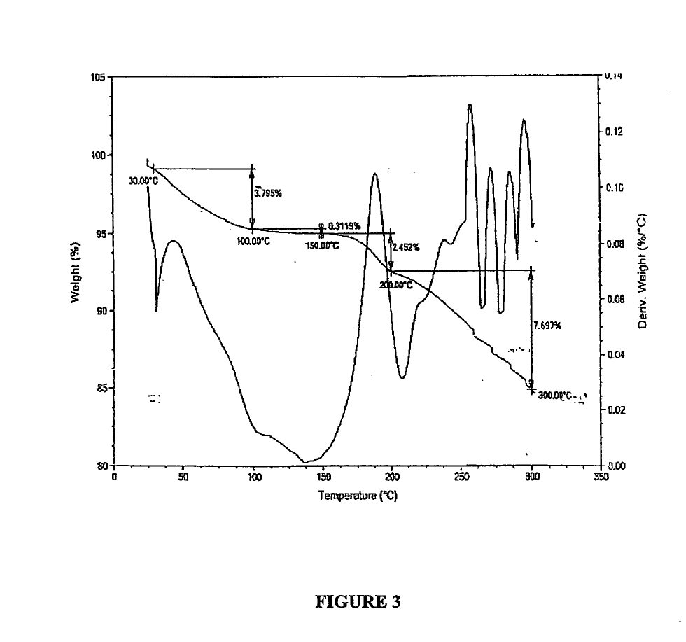

As shown in FIG. 3, the thermogravimetric analysis of polymorph form X of esomeprazole potassium recorded on TGA Q500 V 6.5 in platinum pan with a temperature rise of about 5° C./min in the range of about 30° C. to about 300° C.

In another embodiment, polymorph form X of esomeprazole potassium may be prepared by at least:

(a) providing a solution of esomeprazole in a first solvent;

(b) adding a second solvent to the solution obtained in step (a);

(c) treating the solution of step (b) with a source of potassium;

(d) concentrating the reaction mass of step (c) under vacuum;

(e) adding a suitable anti-solvent; and

(f) recovering esomeprazole potassium in polymorph form X.

In one embodiment, esomeprazole neutral form can be prepared either from the corresponding esomeprazole salts such as potassium, sodium, magnesium, lithium, alkyl ammonium salt and more or less immediately processed through the subsequent steps defined above. If so, the esomeprazole salt as mentioned above can be prepared by any of the methods known in the art and thereafter suspended in a first solvent. Next, the pH can be adjusted to produce esomeprazole in its neutral form. The pH adjustment can be made by the addition of about 1 molar equivalent of a suitable acid HA, preferably as an aqueous solution. Examples of such acid HA comprises but not limited to, all mineral acids that forms a water soluble potassium salt, e.g. hydrochloric acid, and acetic acid. The aqueous phase is thereafter discarded and the organic phase is optionally washed with water or brine. Esomeprazole neutral form is now ready to be used, more or less immediately, in the intermediate steps discussed above.

Suitable first solvents include, but are not limited to, aromatic solvents such as alkyl, aryl, halo substituted benzenes; chlorinated solvents; ketones and the like and mixture thereof. Useful aromatic solvents include toluene, xylene, chlorobenzene, bromobenzene and the like and mixtures thereof. Useful chlorinated solvents include dichloromethane, dichloroethane, chloroform, carbon tetrachloride, perchloroethylene and the like and mixtures thereof. Useful ketones include acetone, iso butyl ketone, methyl isobutyl ketone, and the like and mixtures thereof. In a preferred embodiment, the first solvent is preferably a chlorinated solvent, with dichloroethane being preferred.

Suitable second solvents include, but are not limited to, organic solvents, such as nitriles, e.g., acetonitrile and the like; C₁₋₄ alcohols, e.g., methanol, ethanol, n-butanol, n-propanol, and the like; ketones, e.g., acetone, methyl iso butyl ketone, iso butyl ketone, and the like and mixtures thereof. Preferably, the second solvent S₂ is acetonitrile. The second solvent is preferably added at room temperature, a temperature in the range of from about 25° C. to about 30° C.

Suitable sources of potassium include potassium hydroxide, potassium t-butoxide, potassium methoxide, and the like and mixtures thereof. The source of potassium can be added either as a solution in water or it may be added as a solid to the solution of esomeprazole in a suitable organic solvent. The sequence of addition of water and/or potassium source is not particularly critical. Additionally, the potassium salt formation can be carried out in any known manner, for example, the potassium source can be added into an esomeprazole solution or an esomeprazole solution may be added to the potassium source.

A suitable anti-solvent is added to the residue obtained from step d of a process of the present invention. Suitable anti-solvents include, but are not limited to, cyclic hydrocarbons, acyclic hydrocarbons and the like and mixtures thereof. Representative examples of such anti-solvents include n-pentane, n-hexane, n-heptane, n-octane, cycloheptane, cyclohexane, and the like and mixtures thereof. Preferably, the anti-solvent is n-heptane.

The esomeprazole potassium recovered using the process of the present invention is substantially in polymorph form X. Polymorph form X of esomeprazole potassium can be recovered by, for example, crystallizing the solution to produce substantially pure esomeprazole potassium crystals; and isolating the crystals by techniques known in the art, e.g., filtration, to obtain polymorph form X of esomeprazole potassium. If desired, the isolated crystals can then be dried. The temperature during stirring can range from about 20° C. to about 35° C. The resulting solid can then be filtered, washed with an organic anti-solvent, e.g., n-heptane, to provide crystals of esomeprazole potassium, and dried using a conventional drying process, as appropriate.

The esomeprazole potassium in polymorph form X thus obtained can contain about 1% to about 8% of moisture by weight, as measured by the Karl Fisher method, and preferably from about 3% to about 6% of moisture by weight.

In another embodiment, polymorph form X of esomeprazole potassium may be prepared by at least:

(a) providing a solution of esomeprazole in a water immiscible organic solvent;

(b) adding a source of potassium to the solution to obtain a biphasic reaction mixture;

(c) separating the aqueous layer from the biphasic reaction mixture and removing water thereof to obtain a residue;

(d) optionally subjecting the residue to stripping to remove water; and

(e) recovering esomeprazole potassium in polymorph form X.

Suitable water immiscible organic solvents include, but are not limited to chlorinated solvents, aromatic hydrocarbons, ester-containing solvents and the like and mixtures thereof. Suitable chlorinated solvents include, but are not limited to, dichloromethane, chloroform, carbon tetrachloride, perchloroethylene, chlorobenzene, dichlorobenzene and the like and mixtures thereof. Suitable aromatic hydrocarbons include, but are not limited to, toluene, xylene, and the like and mixtures thereof. Suitable ester-containing solvents include, but are not limited to ethyl acetate, methyl acetate, isopropyl acetate and the like and mixtures thereof. Preferred Suitable water immiscible organic solvents are chlorinated solvents and the most preferred water immiscible organic solvent is dichloromethane.

Suitable sources of potassium include potassium hydroxide, potassium t-butoxide, potassium methoxide, and the like and mixtures thereof. The source of potassium can be added either as a solution in water or it may be added as a solid to the solution of esomeprazole in a suitable organic solvent. The sequence of addition of water and/or potassium source is not particularly critical. Additionally, the potassium salt formation can be carried out in any known manner, for example, the potassium source can be added into an esomeprazole solution or an esomeprazole solution may be added to the potassium source.

Removal of water from the aqueous layer can be carried out by employing any conventional technique such as, for example, concentration by subjecting the aqueous layer to heating, spray drying, freeze drying, evaporation on rotary evaporator under vacuum, agitated thin film evaporator (ATFE) and the like.

Stripping can be carried out by any conventional means by subjecting to treating with a water miscible solvent such as dissolving/slurrying in the solvent and removing the solvent by conventional techniques. Suitable water miscible solvents include, but are not limited to, acetone, methyl ethyl ketone, methyl isobutyl ketone, diethyl ketone and the like and mixtures thereof. The esomeprazole potassium salt in polymorph form X thus obtained can be dried using conventional drying process, as appropriate.

Esomeprazole potassium in polymorph form X obtained by the processes of the present invention contains about 1% to about 8% of moisture by weight, as measured by Karl Fisher method, and preferably from about 3% to about 6% of moisture by weight.

Certain operational steps are well known in the art and, unless otherwise indicated, any known method for performing these functions may be used in the processes of this invention. For example, solvents may be removed by distillation in atmosphere, under vacuum, spray drying, freeze-drying, evaporation on rotary evaporator under vacuum, agitated thin film evaporator (ATFE) and the like. Stirring means any method for blending or mixing a reaction mixture.

The phrase “more or less immediately” used in the present invention is to be understood to mean that the subsequent step or action shall be performed at such a time to avoid degradation of the active compound. The subsequent step can be performed considerably later in time provided that due care has been taken to avoid degradation of the active compound.

The processes of the present invention advantageously allow for the esomeprazole potassium salt in polymorph form X to be prepared directly from the corresponding salts of esomeprazole in high yield and good quality. Additional advantages include high reproducibility, and good process ability including safety.

The processes of the present invention advantageously provide esomeprazole potassium in polymorph form X in relatively high purity, e.g., a purity of greater than or equal to about 96% as measure by HPLC, preferably greater than or equal to about 99%, and more preferably greater than or equal to about 99.5%.

Yet another embodiment of the present invention is directed to pharmaceutical compositions containing at least a therapeutically effective amount of polymorph form X of esomeprazole potassium of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The polymorph form X of esomeprazole potassium of the present invention may also be administered as suppositories, ophthalmic ointments and suspensions, and parenteral suspensions, which are administered by other routes. The dosage forms may contain polymorph form X of esomeprazole potassium of the present invention as is or, alternatively, may contain the polymorph form X of esomeprazole potassium of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the polymorph form X of esomeprazole potassium of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

In one embodiment, the polymorph form X of esomeprazole potassium disclosed herein for use in the pharmaceutical compositions of the present invention can have a D₅₀ and D₉₀ particle size of less than about 300 microns, preferably less than about 200 microns, more preferably less than about 150 microns, still more preferably less than about 50 microns and most preferably less than about 10 microns. It is noted the notation D_{X} means that X % of the particles have a diameter less than a specified diameter D. Thus, a D₅₀ of about 400 microns means that 50% of the micronized particles in a composition have a diameter less than about 400 microns. The term “micronization” used herein means any process or methods by which the size of the particles is reduced. For example, the particle sizes of the polymorph form X of esomeprazole potassium of the present invention can be obtained by any milling, grinding, micronizing or other particle size reduction method known in the art to bring the solid state form of the polymorph form X of esomeprazole potassium of the present invention into any of the foregoing desired particle size range. As also used herein, polymorph form X of esomeprazole potassium particles with reduced size are referred to as “micronized particles of polymorph form X of esomeprazole potassium” or “micronized polymorph form X of esomeprazole potassium”.

Actual dosage levels of polymorph form X of esomeprazole potassium of the present invention may be varied to obtain an amount of polymorph form X of esomeprazole potassium of the present invention that is effective to obtain a desired therapeutic response for a particular composition and method of administration. The selected dosage level therefore depends upon such factors as, for example, the desired therapeutic effect, the route of administration, the desired duration of treatment, and other factors. The total daily dose of the compounds of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc. The pharmaceutical compositions herein can be formulated in any release form, e.g., immediate release, sustained release, controlled release, etc.

The following examples are provided to enable one skilled in the art to practice the invention and are merely illustrative of the invention. The examples should not be read as limiting the scope of the invention as defined in the features and advantages.

### Example 1

### Preparation of polymorph form X of 5-Methoxy-2-[(S)-(4-methoxy-3,5-dimethyl-2-pyridinylmethyl)sulfinyl]-1H-benzimidazole potassium

The potassium salt of esomeprazole (10 g) was dissolved in water (20 ml), which was then followed by addition of methylene chloride (20 ml) at 20 to 30° C. The pH of the aqueous layer was adjusted to about 7 to 8.5 by the addition of acetic acid. The aqueous layer was separated and extracted with methylene chloride (20 ml). The organic layer was combined and washed with water. Acetonitrile (50 ml) was added to the organic layer, which was allowed to cool, and potassium hydroxide solution (1.4 grams of potassium hydroxide dissolved in 1.4 ml of water) was added. The resulting solution was stirred for 60 minutes and concentrated under vacuum below 40° C. This was followed by the addition of 50 ml of acetone to the resulted residue and the solvent was again evaporated under vacuum. 100 ml of heptane was added to the resulted residue and the reaction mass were stirred overnight. The resulting mass was filtered and dried to provide the potassium salt of esomeprazole. Yield: 5 grams

The XRD of the final product is set forth in FIG. 1 and was recorded and identified as polymorph form X of esomeprazole potassium. The XRD confirms that form X has a characteristic d-spacing value at 16.6 and a 2-Theta value of 5.3.

HPLC purity: 99.5%

### Example 2

### Preparation of polymorph form X of 5-Methoxy-2-[(S)-(4-methoxy-3,5-dimethyl-2-pyridinylmethyl)sulfinyl]-1H-benzimidazole potassium

The sodium salt of esomeprazole (10 g) was dissolved in water (20 ml), which was then followed by addition of methylene chloride (20 ml) at 20 to 30° C. and pH of the aqueous layer was adjusted about 7 to 8.0 by addition of acetic acid. The aqueous layer was separated and extracted with methylene chloride (20 ml). The organic layer was combined and followed by washing with water. Acetonitrile (50 ml) was added to the organic layer, which was allowed to cool, followed by addition of potassium hydroxide solution (1.5 grams of potassium hydroxide dissolved in 1.5 ml of water). The resulting solution was stirred for 60 minutes and concentrated under vacuum below 40° C. Acetone (50 ml) was added to the resulted residue and the solvent was again evaporated under vacuum. Heptane (100 ml) was added to the resulting residue and the reaction mass was stirred overnight. The resulting mass was filtered and dried to obtain the potassium salt of esomeprazole. Yield: 5.6 grams

The XRD of the final product is set forth in FIG. 1 and was recorded and identified as polymorph form X of esomeprazole potassium. The XRD confirms that form X has a characteristic d-spacing value at 16.6 and 2-Theta value 5.3.

HPLC purity: 99.5%

### Example 3

### Preparation of 5-Methoxy-2-[(S)-(4-methoxy-3,5-dimethyl-2-pyridinylmethyl)sulfinyl]-1H-benzimidazole potassium

Esomeprazole (10 g) was dissolved in methylene dichloride (50 ml). A solution of potassium hydroxide solution (1.46 grams of potassium hydroxide dissolved in 25 ml of water) was added to the solution and stirred for 120 minutes. The aqueous layer was separated and concentrated in a rotary evaporator under vacuum below 40° C. To the residue, acetone (200 ml) was added and stirred. Acetone was distilled out completely in a rotary evaporator under vacuum to obtain esomeprazole potassium solid (5.5 g). XRD confirmed the esomeprazole potassium was form X having a characteristic d-spacing value at 16.6 and 2-Theta value 5.3.

HPLC purity: 99.5%

### Example 4

### Preparation of 5-Methoxy-2-[(S)-(4-methoxy-3,5-dimethyl-2-pyridinylmethyl)sulfinyl]-1H-benzimidazole potassium

Esomeprazole (10 g) was dissolved in methylene dichloride (50 ml). A solution of potassium hydroxide solution (1.46 grams of potassium hydroxide dissolved in 25 ml of water) was added to the solution and stirred for 120 minutes. The aqueous layer was separated and spray dried in a spray drier to obtain esomeprazole potassium solid (3.8 gm). XRD confirmed the esomeprazole potassium was Form X having a characteristic d-spacing value at 16.6 and 2-Theta value 5.3.

HPLC purity: 99.55%

Experimental data:

The moisture content as measured by the Karl Fisher method for three batches of esomeprazole potassium in polymorph form X of the present invention is set forth below in Table II.

TABLE II

| Batch | Moisture Content |
|---|---|
| 1 | 3.9% w/w |
| 2 | 5.2% w/w |
| 3 | 5.4% w/w |

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

1-25. (canceled)

26. A potassium salt of esomeprazole of form X.

27. The potassium salt of esomeprazole of claim 26, having a differential scanning calorimetric (DSC) thermogram substantially in accordance with FIG. 2.

28. The potassium salt of esomeprazole of claim 26, having a purity of at least about 96%.

29. The potassium salt of esomeprazole of claim 26, having a purity of at least about 99%.

30. A pharmaceutical composition comprising the potassium salt of esomeprazole of claim 26, and a pharmaceutically acceptable carrier, diluent or excipient.

31. The pharmaceutical composition of claim 30, wherein the potassium salt of esomeprazole has a D₅₀ particle size of less than about 300 microns.

32. The pharmaceutical composition of claim 30, wherein the potassium salt of esomeprazole has a D₅₀ particle size of less than about 150 microns.

33. A process for preparing esomeprazole potassium of form X, the process comprising:
(a) providing a solution of esomeprazole in a first solvent;
(b) adding a second solvent to the solution obtained in (a);
(c) treating the solution of (b) with a source of potassium;
(d) concentrating the product of step (c) under vacuum;
(e) adding an anti-solvent; and
(f) recovering esomeprazole potassium of form X.

34. The process of claim 33, wherein the first solvent is dichloromethane, the second solvent is acetonitrile and the anti-solvent is n-heptane.

35. A process for preparing a form X of esomeprazole potassium, the process comprising:
(a) providing a solution of esomeprazole in a water immiscible organic solvent;
(b) adding a source of potassium to the solution in step (a) to obtain a biphasic reaction mixture;
(c) separating the aqueous layer from the biphasic reaction mixture in step (b) and removing water therefrom to obtain a residue; and
(d) recovering esomeprazole potassium of form X.

36. The process of claim 35, wherein the water immiscible organic solvent is a chlorinated solvent.

37. The process of claim 36, wherein the chlorinated solvent is selected from the group consisting of dichloromethane, chloroform, carbon tetrachloride, perchloroethylene, chlorobenzene, dichlorobenzene and mixtures thereof.

38. The process of claim 37, wherein the chlorinated solvent is dichloromethane.
